(19) United States
(12) Patent Application Publication
(10) Pub. No.: US 2009/0318824 A1
(43) Pub. Date: Dec. 24, 2009

(54) NEURALPROBE AND METHODS FOR MANUFACTURING SAME

(75) Inventors: Toshikazu Nishida, Gainesville, FL (US); Huikai Xie, Gainesville, FL (US); Erin E. Patrick, Vero Beach, FL (US); Justin C. Sanchez, Newberry, FL (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(73) Assignee: University of Florida Research Foundation Inc., Gainesville, FL (US)

(21) Appl. No.: 11/915,987

(22) PCT Filed: Jun. 1, 2006
(86) PCT No.: PCT/US06/21338

§ 371 Date: Jul. 14, 2008

Related U.S. Application Data
(60) Provisional application No. 60/686,275, filed on Jun. 1, 2005.

(51) Int. Cl. A61B 5/0478; H01L 21/465

(52) U.S. Cl. 600/544; 438/700; 257/E21.485

(57) ABSTRACT

A neural probe and method of fabricating same are provided. The probe comprises a plurality of frames connected to each other and to a substrate by respective bimorphs. A probe base is connected by another bimorph to the frames. A probe tip extends from the probe base. The probe can achieve a large vertical motion and out-of-plane curling. The probe can operate according to three modes. The first mode pertains to a large-signal motion for tuning in single-unit neuronal activity. The second pertains to a small-signal motion with lock-in amplifier that increases SNR. The third pertains to burst small-signal motion for clearing tissue responses. Fabrication of a neural probe begins with a processed CMOS chip. Post-CMOS processing incorporates self-aligned selective nickel plating and sacrifices two aluminum layers. The fabrication technique produces a neural probe in which the sensing elements are in close proximity to CMOS circuitry. The fabrication technique obviates the need for post-CMOS masks, alignment, or assembly.

### FIELD OF THE INVENTION

The present invention is related to the field of electronic sensors, and more particularly, to electronic sensors for sensing neuronal activity.

### BACKGROUND OF THE INVENTION

Neural prosthetics are chips that model brain function and that can be implanted in a living organism to replace damaged or dysfunctional portions of the brain or other tissue of the organism's nervous system. For example, a neural prosthetic can comprise an intracranial implant or computer chip that models a brain function so as to replace damaged or dysfunctional brain tissue. As a result of relatively recent advances in neuroscience and bioengineering, there is a likelihood of more biologically realistic mathematical models of the brain and spinal cord functions, as well as silicon and/or photonics-based computational devices that can incorporate such models. In addition, there is a drive for enhanced neuron-silicon interface devices, such as micro-scale electrodes that can provide bi-directional communication between the computational devices and functioning brain tissue.

A neuron-silicon interface device can sense and record neuronal activity, typically with a subdural microelectrode. A subdural microelectrode is a small electrode that can sense an electrical signal, often from a single nerve cell. The subdural microelectrode extends beneath the dura—a tough membrane covering the brain and spinal cord—and above the arachnoid membrane so as to sense neuronal activity. Nerve cells, or neurons, are the primary cells of the nervous system. Nerve cells, in vertebrates, are found in the brain and the spinal cord as well as the nerves and ganglia of the peripheral nervous system. In sensing neuronal activity, the microelectrode typically can be directed to an area where one nerve ends and another begins, sensing impulses that pass over a synapse from one nerve to another.

One yet-to-be-resolved obstacle confronting designers of conventional subdural microelectrode neural prosthetics is the limited control of probe-to-neuron distance owing to the fixed probe length of many conventional devices. Another obstacle is the low signal level that is to be sensed with such a device, the level typically being on the order of only several microvolts. Still another obstacle is the gradual decline in sensitivity of the neural probe that often occurs over time.

Different approaches to these problems have been proposed. These include coating the electrode with a material for affecting the glial response and mitigating tissue inflammation. Proposed devices include multi-site shanks for targeting the columnar cortical structure, microdrive electrodes for “tuning in” cellular activity, and rapid injection probes for minimizing implantation injuries.

These and other approaches, however, have typically failed to adequately address the problems already described concerning conventional devices. There thus remains in the art for an effective and efficient neural probe that overcomes these obstacles and limitations.

### SUMMARY OF THE INVENTION

The present invention provides a neural probe and related methods for manufacturing such a probe. More particularly, one aspect of the invention is a probe that operates according to three modes. The first mode is a large-signal motion mode of operation for “tuning in” single-unit neuronal activity. The second mode is a small-signal motion mode of operation with lock-in amplifier that increases a signal-to-noise ration (SNR). The third mode is a burst small-signal motion mode of operation for clearing tissue responses. By being capable of operating according to these modes, the probe can provide enhanced sensing and recording of neuronal activity.

One embodiment of the invention is a micro-electromechanical system (MEMS) probe for sensing neuronal activity. The probe can include a probe base having at least one preamplifier embedded therein. A bimorph can be mechanically connected to the probe base, the bimorph being capable of flexing in a predetermined direction in response to an applied electrical signal. A probe tip can extend from the probe base, the probe tip containing at least one electrode embedded therein and connected to the at least one preamplifier. Moreover, the probe can have a first mode of operation for large-signal motion in sensing single-unit neural activity, a second mode of operation for small-signal motion to increase a signal-to-noise ratio, and a third mode of operation for burst-type small-signal motion for clearing tissue responses.

According to another embodiment, a micro-electromechanical system (MEMS) probe for sensing neuronal activity can include a first probe frame and a first bimorph for mechanically connecting the first probe frame to a semiconductor substrate, the first bimorph being capable of flexing in a predetermined direction in response to an applied electrical signal. The MEMS probe, according to this embodiment of the invention, also can include a second probe frame and a second bimorph mechanically connecting the second probe frame to the first probe frame, the second bimorph being capable of flexing in a predetermined direction in response to an applied electrical signal. According to this embodiment, moreover, the MEMS probe can further include a probe base having at least one preamplifier embedded therein and a third bimorph mechanically connecting the probe base to the second bimorph, the third bimorph being capable of flexing in a predetermined direction in response to an applied electrical signal. Additionally, according to-this embodiment, the MEMS probe can include a probe tip extending from the probe base, the probe tip containing at least one electrode embedded therein and connected to the at least one preamplifier.

Another aspect of the invention is a method of manufacturing a neural probe from a processed CMOS chip. Post-CMOS processing according to the invention can incorporate self-aligned selective nickel plating and sacrifices two aluminum layers. The fabrication technique can produce a neural probe in which the sensing elements are in close proximity to CMOS circuitry. The fabrication technique, moreover, can eliminate the need for post-CMOS masks, alignment, or assembly.

### BRIEF DESCRIPTION OF THE DRAWINGS

There are shown in the drawings, embodiments which are presently preferred. It is to be understood, however, that the invention is not limited to the precise arrangements and instrumentalities shown in the drawings.

FIG. 1 is a top-view schematic diagram of a neural probe, according to one embodiment of the present invention.

FIG. 2 is a schematic diagram of a probe tip and probe base of a neural probe, according to another embodiment of the invention.

FIG. 3 is a cross-sectional diagram of the neural probe and portions of the neural base illustrated in FIG. 2.

FIGS. 4A and 4B are schematic diagrams illustrating displacements of respective portions of a neural probe, according to still another embodiment of the invention.

FIG. 5 is an ordered sequence of cross-sectional views of a CMOS wafer or chip as it is fabricated into a neural probe through a series of processing steps, according to yet another embodiment of the invention.

FIG. 6 is a schematic diagram of an array of probe tips for a neural probe, according to still another embodiment of the invention.

FIG. 7 is a perspective view of a neural probe including an electrostatic comb device, according to yet another embodiment of the invention.

FIG. 8 is a schematic diagram of package containing a CMOS-MEMS neural probe, according to still another embodiment of the invention.

FIG. 9 is a schematic diagram of neural probe including a plurality of thermal fuses, according to yet another embodiment of the invention.

### DETAILED DESCRIPTION

The invention provides a neural probe and related methods for fabricating a neural probe. The neural probe, more particularly, can comprise a micro-machined moveable neural probe that operates according to three distinct modes of operation. The first mode pertains to large-signal motion for “tuning in” to single-unit neuronal activity. The second mode pertains to small-signal motion with an amplifier lock-in to increase signal-to-noise ratios. The third mode of operation pertains to burst small-signal motion for clearing tissue responses.

Through these distinct modes of operation, a neural probe according to the invention can overcome limitations inherent in conventional devices such as the limited control of probe-to-neuron distance due to the fixed probe length of various types of conventional devices. The neural probe according to the invention can also overcome limitations occurring as a result of the low level—typically only a few microvolts—of signals that are sensed and recorded with a neural probe. Additionally, a neural probe according the invention can mitigate the decline in probe sensitivity that often occurs with conventional devices.

A method of fabrication according to the invention also provides unique advantages. According to one embodiment, fabrication of a neural probe comprises a post-complementary metal oxide semiconductor (CMOS) processing sequence. The sequence can incorporate self-aligned selective nickel plating of electrodes comprising a probe tip and sacrifice of aluminum or other sacrificial layers. The sacrifice of two aluminum layers provides a mechanism for fabricating a neural probe having a probe tip that is in close proximity to a CMOS circuit. This is achieved without the need for post-CMOS masks, alignments, or assembly.

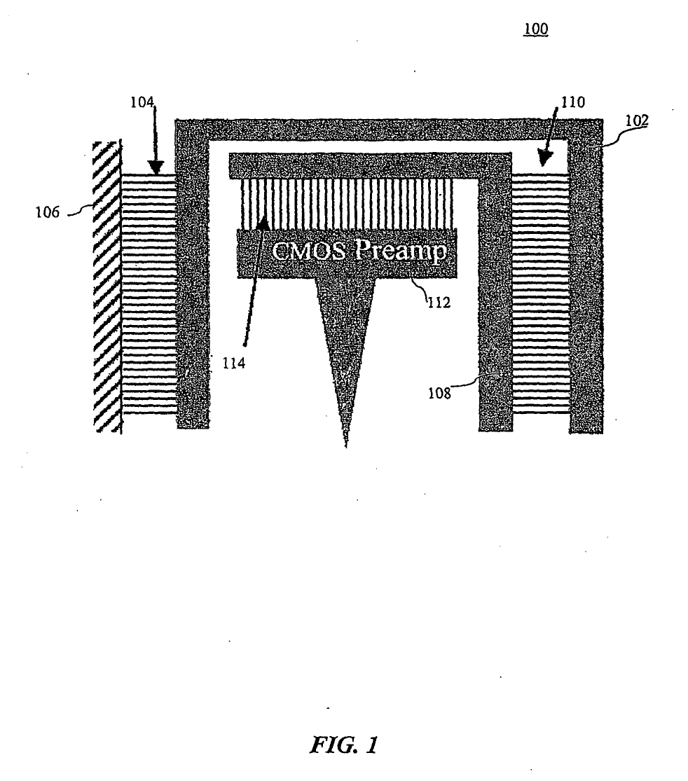

FIG. 1 provides an integrated CMOS micro-electromechanical system (MEMS) neural probe 100, according to one embodiment of the invention. The neural probe 100 illustratively includes a first probe frame 102 and a corresponding first bimorph 104 that mechanically connects the first probe frame 102 to a semiconductor substrate 106. The first bimorph 104, moreover, is capable of flexing in a predetermined direction in response to an applied electrical signal.

Illustratively, the neural probe 100 further includes a second probe frame 108 and a second bimorph that mechanically connects the second probe frame to the first probe frame 102. The second bimorph is also capable of flexing in a predetermined direction in response to an applied electrical signal. The neural probe 100 also illustratively includes a probe base 112 having one or more preamplifiers, such as a CMOS preamplifier, embedded therein. As used herein, the term embedded denotes a component that is disposed on or contained within the object in which it is embedded.

The neural probe 100 illustratively includes a third bimorph 114 that mechanically connects the probe base 112 to the second probe frame 108, the third bimorph also being capable of flexing in a predetermined direction in response to an applied electrical signal. Extending from the probe base 112 is a probe tip 116 having one or more electrodes that are embedded in the probe tip and that connect to the one or more preamplifiers embedded in the probe base.

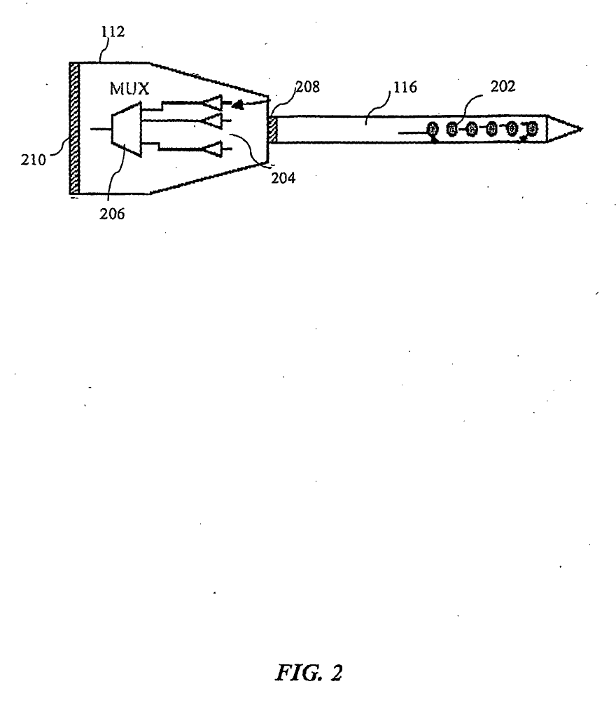

Referring additionally to FIG. 2, the probe tip 116 illustratively comprises a plurality of electrodes 202 at the distal end of the probe tip. Each of the electrode 202 is capable of conveying a sensed signal to the plurality of preamplifiers 204 embedded in the probe base 112. Illustratively, each of the plurality of preamplifiers 204 connects to a multiplexer 206. A first thermal/electrical isolation region 208 is illustratively disposed between the probe tip 116 and the probe base 112. A second thermal/electrical isolation region 210 is disposed on the opposing end of the probe base.

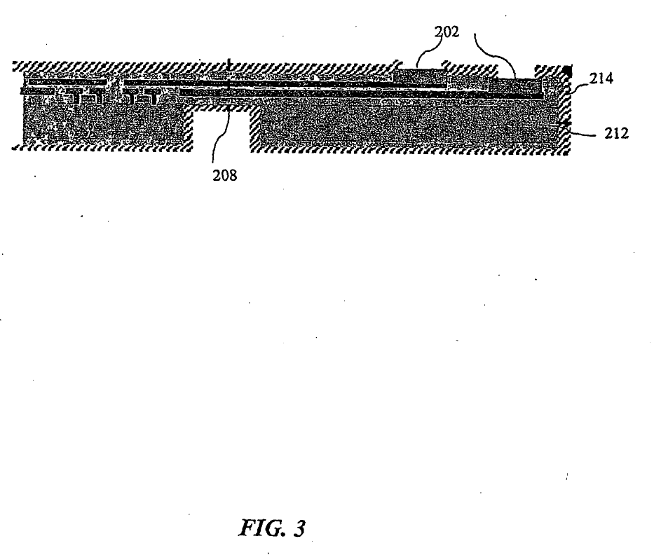

Referring additionally to FIG. 3, a cross-sectional view of the probe tip 116 and a portion of the probe base 112, including the first thermal/electrical isolation region 208 in between, shows that the electrodes 202 can be embedded in a separate layer overlaying a silicon layer. The silicon layer can be approximately 45 micrometers (am) thick. A coating can extend over the probe tip 116 and portion of the probe base 112, with regions overlying the electrodes etched away to expose the respective electrodes, as further illustrated. The coating can comprise a biocompatible material, as will be readily understood by one of ordinary skill in the art.

Neural signals sensed by the neural probe 100 typically have frequencies in the range of 100 hertz (Hz) to a few kHz. These neural signals also typically have DC offsets. AC coupling with a large time constant optionally can be used to reduce or eliminate the DC offset in neural signals sensed by the neural probe 100. Accordingly, each of the plurality of electrode 202 can be AC coupled to a separate one of the plurality of preamplifiers 204. The preamplifiers 204, more particularly, can each comprise an operational transconductance amplifier (OTA). The multiplexer 206 can comprise an analog multiplexer for time multiplexing the signals conveyed by the plurality of preamplifiers 204 connected thereto.

Each electrode can comprise a metal, such as nickel, that acts as the gate of a metal-oxide semiconductor field-effect transistor (MOSFET). Large-area pMOS transistors can be used for sensing to mitigate flicker noise, 1/ƒ. Moreover, a chopper stabilization technique, as understood by one of ordinary skill in the art, can be employed to further reduce the flicker noise. Common mode feedback can be used to reduce the amplifier offset. The signals can be modulated for further amplification. According to another embodiment, floating gate transistors can be utilized in the probe tip 116 to directly sense neuron signals.

The first and second biomorphs can each comprise a silicon beam with an aluminum layer on one surface of the silicon beam. When an electrical current is applied, the temperature of each layer—the silicon and aluminum—increases and the different thermal coefficients of the respective layers cause a flexing or bending like a bimetal, resulting in a lateral motion. Thus, the first and second bimorphs serve as a pair of folded thermal actuators that form a planar platform for supporting the probe tip 116. The cascading of the probe tip 116. and a low-voltage differential (LVD) actuator generates a large vertical displacement in response to a signal (e.g., electrical current).

The third bimorph curls 90 degrees. An offset from 90 degrees can be compensated for with an embedded polysilicon heater. This novel design provides for large vertical motion and out-of-plane curling for vertical orientation and displacement. Unlike electrostatic actuation, which consumes low power but produces small forces, thermoelastic actuation can generate larger forces. The force of the thermoelastic actuator can be designed to exceed the one milli-Newton (1 mN) of force typically needed for insertion of the neural probe 100 into the cranial matter of a subject.

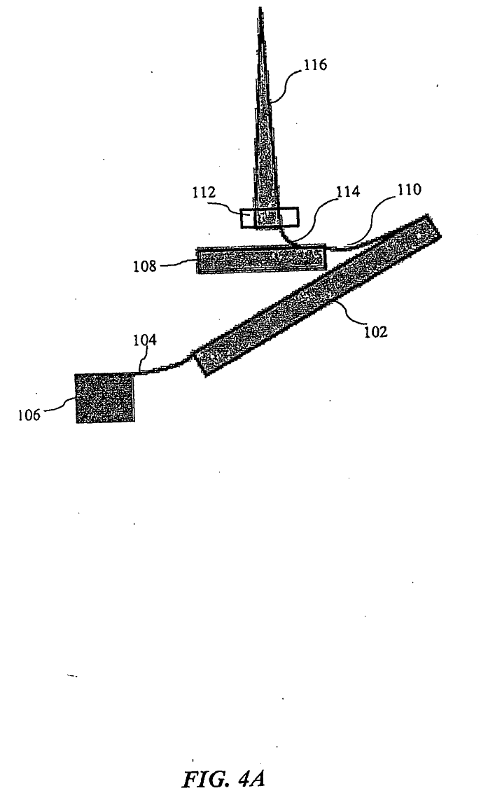

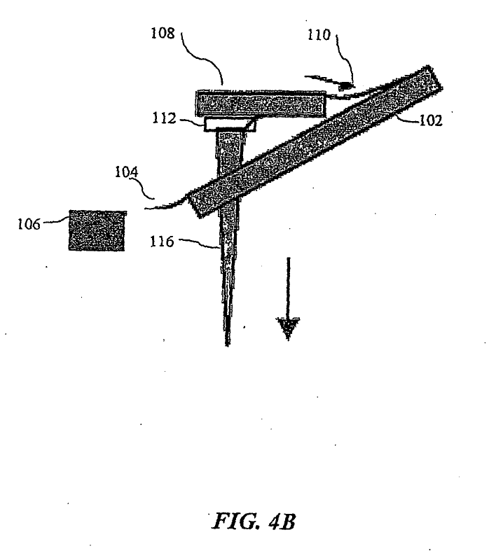

FIGS. 4A and 4B schematically illustrate lateral movements of the probe tip 116 relative to a planar platform. As shown and as discussed more particularly in the context of fabrication of the neural probe 100, the vertical displacement can be upward or downward depending on the orientation of the silicon and aluminum layers relative to each other.

A tilt angle for a bimorph of approximate length 200 micrometers (μm) is at or near 45 degrees. To achieve a 2 millimeter (mm) vertical stroke, the frame length L can be determined to be approximately 2.8 mm: L=2/sin θ. A first-order calculation, therefore, suggests that the force density of the bimorph actuators is about 12 nN per temperature change in degrees Kelvin, K, per bimorph width, W μm. For a 3 mm=3000 μm bimorph width, the force density per unit temperature change is about 36 μN/K. The local temperature increase needed to exert 1 mN force is about 30K.

Adverse temperature effects can be mitigated by with a micro-scaled device that reduces or minimizes the heat required for actuation of the device. Heating of the probe can be minimized by (1) designing the actuator to minimize thermal gradient required for actuation and (2) design of thermal resistances to minimize conduction of heat to probe. Furthermore, by increasing the width of the bimorph, the force density is increased, thus reducing the necessary local temperature rise. Similarly, designing thermal resistances to minimize heat conduction to the probe also can mitigate adverse temperature effects.

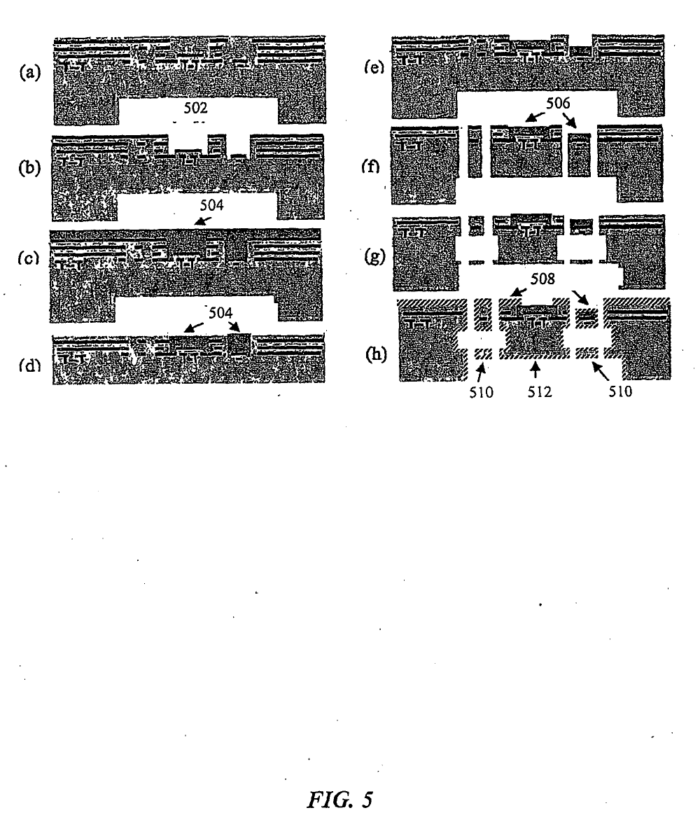

FIG. 5 schematically illustrates the fabrication of a neural probe through a succession of processing steps 500. The process starts with a CMOS wafer or chip. Initially, at step (a), a silicon membrane is formed by backside etching of the wafer or chip followed by plasma enhanced chemical vapor deposition (PECVD) oxide passivation. Thus is formed an oxide layer 502 on the backside of the wafer or chip as illustrated. Subsequently, at step (b), shallow cavities for neural electrodes are formed by performing an anisotropic oxide etch from the front side of the wafer or chip. Aluminum is first used as the etching mask and is then removed. The aluminum on the bottom of the cavities is protected by a spin-on photoresist 504, as illustrated at steps (c) and (d). The photoresist is then removed at step (e), and nickel 506 is selectively electroplated on the cavity regions.

Pretreatment using Zincate can be optionally performed if needed. The side growth of the nickel layer during electroplating is used to cover all areas of exposed aluminum. Next, at step (f), an anisotropic etch is performed, and then, deep silicon etching using aluminum as an etching mask is preformed. Another anisotropic oxide etch is subsequently performed to etch through the backside oxide layer. Then, after the top aluminum layer is removed, an isotropic silicon etch is subsequently performed to undercut the silicon beneath narrow beams at step (g). Finally, a biocompatible coating layer is applied to the entire structure at step (h).

According to one embodiment, parylene-C coating and/or oxide/nitride/oxide dielectric 508 can optionally be used. Parylene-C is frequently used as an insulating material for microelectrode implants. Histological studies show normal neurons adjacent to implanted electrodes coated with parylene-C, suggesting that it is well suited for such purposes. Parlyene-C has also been shown to be a good dielectric, and the hydrophobic surface of parlyene-C insulation may discourage fibrosis on the electrode, as well as the development of excess tissue in the region.

In the context of fabrication of the neural probe, note that thermal fuses can be used to hold the entire structure in a desired plane. As described more particularly below, this use of thermal fuses in packaging can decrease difficulties encountered with conventional packaging techniques, and accordingly, increase fabrication yields.

A key aspect is the sacrificing of two metal layers. Sacrificing two metal layers obviates the need for post-CMOS masks, alignment, or assembly. Moreover, the CMOS circuits are integrated in close proximity to the probe. The integrated probe tip region 512 can curl in a vertical direction. The curl can be achieved using different thermal coefficients of expansion for the particular bimorph materials of aluminum and oxide. The thermal coefficient of expansion of aluminum is considerably larger than that of silicon oxide: 23 μstrain/K for aluminum as opposed to 0.7 μstrain/K for silicon oxide. By disposing aluminum either on top or bottom of the stack comprising the bimorph, the beams comprising the bimorphs can be made to curl up or down.

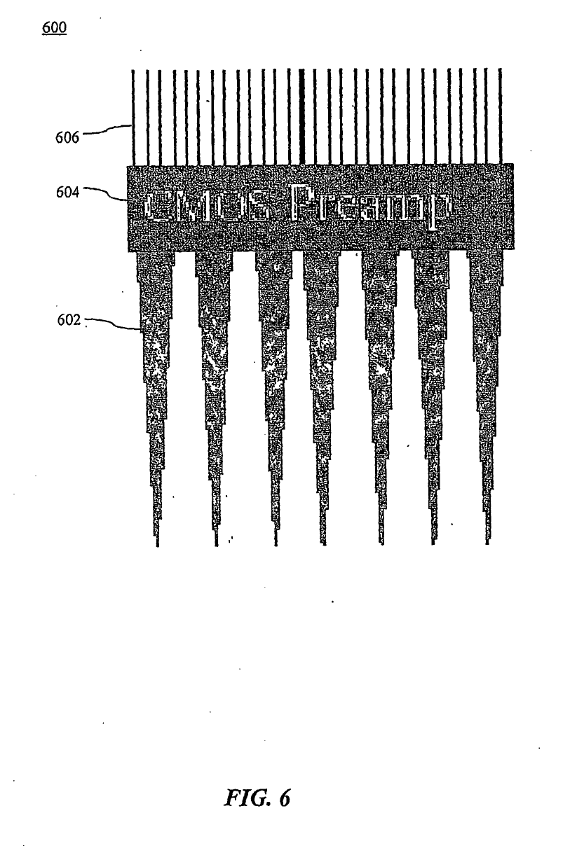

Still another embodiment of the invention is a probe array. As illustrated in FIG. 6, the probe array 600 comprises a plurality of probe tips 602 that extend from a probe base 604. A bimorph 606 mechanically connects the probe base to other portions of a neural probe similar to those already described.

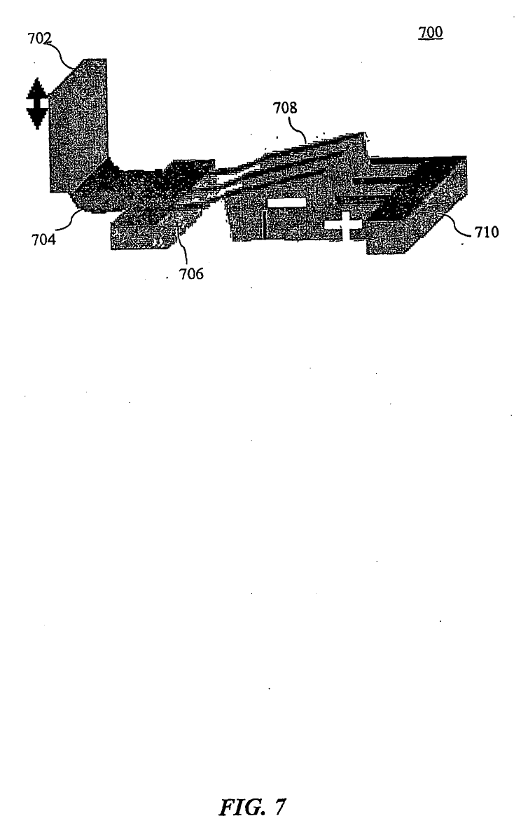

FIG. 7 is a perspective view of neural probe 700 according to yet another embodiment of the invention. The neural probe includes a probe tip 702 connected via a bimorph 704 to a frame 706. A second bimorph connects the frame 706 to a second frame 708, which defines the rotor of a rotor-stator electrostatic comb. The rotor illustratively includes distinct fingers, each mechanically connected to a unique one of a plurality of beams that define the second bimorph. Illustratively, the distinct fingers of the second frame are interdigitated with respective ones of a plurality of beams of a third frame 710 that defines the stator of the electrostatic comb. The vertical electrostatic comb drive can be embedded to support the probe tip for sensing and for fine actuation control. The electrostatic actuation can produce about 10 μm of vertical motion at an oscillation frequency of a few kilohertz.

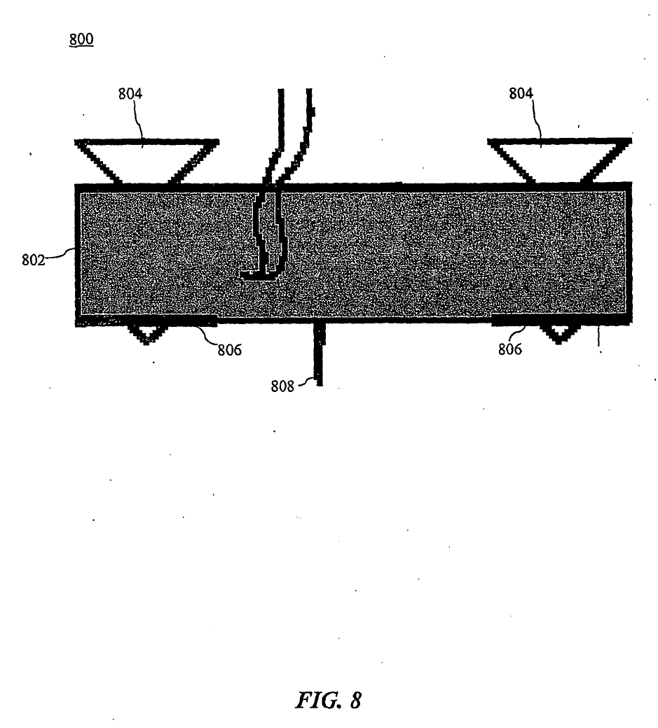

As illustrated by the schematic diagram of FIG. 8, a neural probe 800 according to the invention can be contained within a discrete package 802. The package 802 can comprise a material that has high thermal conductivity and that is electrically insulating, such as can be provided by anodized aluminum. The package can attach to another object with one or more fasteners 804. Thermal insulation 806 can overlie portions of the package 802. One or more probe tips 808 can extend from the package 802. Thus, a CMOS-MEMS moveable neural probe chip according to the invention can be flush mounted in the package, as illustrated.

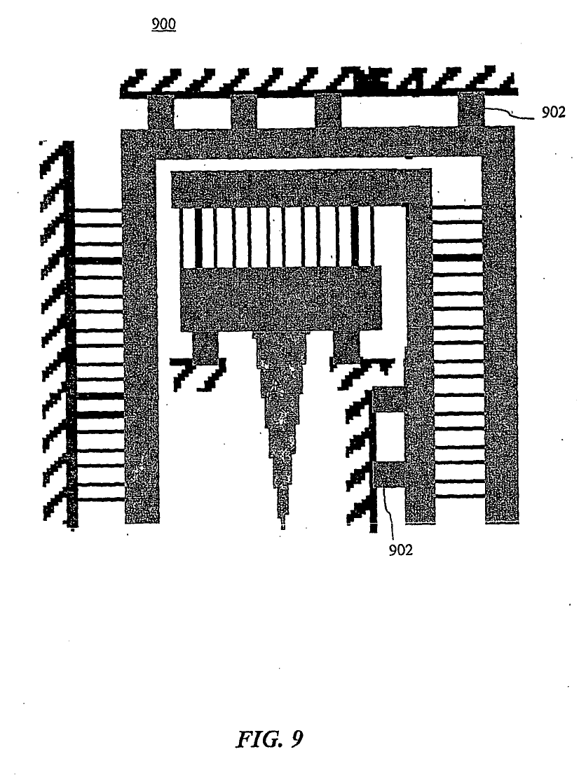

FIG. 9 further illustrates the packaging of the CMOS-MEMS neural probe 900. To facilitate high-yield packaging, the neural probe can be held in place by thin, short beams. The thin, short beams can comprise polysilicon heaters embedded as thermal fuses 902. Accordingly, when the neural probe 900 is acceptably positioned within the package, a current can be applied to the fuses 902, the current being of a sufficient magnitude to blow the short beams. This manner of packaging the neural probe 900 can be expected to ease the difficulties confronted with conventional packaging of such devices and, accordingly, can be expected to increase the yield of fabrication.

This invention can be embodied in other forms without departing from the spirit or essential attributes thereof. Accordingly, reference should be made to the following claims, rather than to the foregoing specification, as indicating the scope of the invention.

We claim:

1. A micro-electromechanical system (MEMS) probe for sensing neuronal activity, the probe comprising:
a probe base having at least one preamplifier embedded therein
a bimorph mechanically connected to the probe base, the bimorph being capable of flexing in a predetermined direction in response to an applied electrical signal;
a probe tip extending from the probe base, the probe tip containing at least one electrode embedded therein and connected to the at least one preamplifier; and
the probe having a first mode of operation for large-signal motion in sensing single-unit neural activity, a second mode of operation for small-signal motion to increase a signal-to-noise ratio, and a third mode of operation for burst-type small-signal motion for clearing tissue responses.

2. The probe of claim 1, wherein the probe further comprises a first probe frame and the bimorph comprises a first bimorph, and wherein the probe further comprises a second probe frame and a second bimorph that mechanically connects the second probe frame to the first probe frame.

3. The probe of claim 1, wherein the at least one electrode comprises a plurality of electrodes, and wherein the at least one preamplifier comprises a plurality of preamplifiers, each of the preamplifiers comprising an operational transconductance amplifier that is AC coupled to a unique one of the electrodes.

4. The probe of claim 3, wherein the probe base further comprises an analog multiplexer embedded therein and connected to the plurality of preamplifiers for time multiplexing analog signals received.

5. The probe of claim 4, further comprising a plurality of MOS-bipolar pseudoresistor elements connected to each of the plurality of preamplifiers to mitigate a DC offset of a neural signal.

6. The probe of claim 5, wherein each of the plurality of electrodes comprises the gate of a metal-oxide semiconductor field-effect transistor.

7. The probe of claim 5, wherein the probe tip further implements a chopper stabilization technique to further mitigate flicker noise.

8. The probe of claim 5, wherein each one of the plurality of electrodes is connected to a pMOS transistor.

9. The probe of claim 1, wherein further comprising a thermally conductive package encasing the probe base, bimorph, and probe tip.

10. A micro-electromechanical system (MEMS) probe for sensing neuronal activity, the probe comprising:
a first probe frame;
a first bimorph for mechanically connecting the first probe frame to a semiconductor substrate, the first bimorph being capable of flexing in a predetermined direction in response to an applied electrical signal;
a second probe frame;
a second bimorph mechanically connecting the second probe frame to the first probe frame, the second bimorph being capable of flexing in a predetermined direction in response to an applied electrical signal;
a probe base having at least one preamplifier embedded therein;
a third bimorph mechanically connecting the probe base to the second bimorph, the third bimorph being capable of flexing in a predetermined direction in response to an applied electrical signal; and
a probe tip extending from the probe base, the probe tip containing at least one electrode embedded therein and connected to the at least one preamplifier.

11. The probe of claim 10, wherein the first and second bimorphs comprise a pair of folded thermal actuators for forming a flat platform for effecting large vertical displacements of the probe tip in response to an electrical signal.

12. The probe of claim 11, wherein the third bimorph flexes approximately ninety degrees (90°).

13. The probe of claim 12, further comprising an embedded polysilicon for compensating an offset from the approximately ninety degrees.

14. The probe of claim 10, wherein the at least one preamplifier comprises a plurality of operational transconductance amplifiers, and further comprising an analog multiplexer for time multiplexing signals received from the operational transconductance amplifiers.

15. A method of fabricating a neural probe, the method comprising:
(a) forming a silicon membrane by backside etching of a processed CMOS wafer or chip and performing a plasma enhanced chemical vapor deposition (PECVD) oxide passivation;
(b) forming shallow cavities for neural electrodes in the CMOS wafer or chip by performing an anisotropic oxide etch from the front side of the CMOS wafer or chip using a metal as an etching mask;
(c) applying a spin-on photoresist to protect some portions of the metal;
(d) removing the top metal layer except portions of the metal protected by the spin-on photoresist;
(e) removing the photoresist and selectively electroplating cavity regions in the CMOS wafer or chip;
(f) performing an anisotropic etch, deep silicon etching and another anisotropic oxide etch to etch through the backside oxide layer;
(g) performing an isotropic silicon etch to etch silicon beneath narrow beams; and
(h) coating the structure with a biocompatible layer.
